(19) United States
(12) Patent Application Publication
(10) Pub. No.: US 2003/0003066 A1
(43) Pub. Date: Jan. 2, 2003

(54) Methods and compositions for conditioning, treating and coloring the skin and its appendages

(76) Inventors: Rosemarie Nichols, Manhattan Beach, CA (US); Martin J. Weisman, Westlake Village, CA (US)

Correspondence Address: BARNES & THORNBURG, 2600 CHASE PLAZA, 10 LASALLE STREET, CHICAGO, IL, 60603

(21) Appl. No.: 10/054,674

(22) Filed: Jan. 22, 2002

Related U.S. Application Data
(63) Continuation-in-part of application No. 09/932,314, filed on Aug. 17, 2001.
Application No. 09/932,314 is a continuation of application No. 09/665,885, filed on Sep. 20, 2000, now Pat. No. 6,395,263.
Application No. 09/665,885 is a continuation of application No. 09/472,355, filed on Dec. 23, 1999, now Pat. No. 6,203,809.
Application No. 09/472,355 is a continuation of application No. 09/266,469, filed on Mar. 11, 1999, now Pat. No. 6,027,739.
Application No. 09/266,469 is a division of application No. 08/647,837, filed on May 15, 1996, now Pat. No. 6,001,374.
Application No. 08/647,837 is a continuation-in-part of application No. 08/440,780, filed on May 15, 1995, now Pat. No. 5,747,017.

(51) Int. Cl. A61K 7/025

(52) U.S. Cl. 424/64

(57) ABSTRACT

Cosmetic compositions for coloring, conditioning, enhancing skin and its appendages are based on a film forming agent to which coloring, enhancing, and moisturizing agents may be added. Compositions also include waxless lip balm with and without color, and enhancing agents with and without color. Because the compositions are not readily removable by normal wear and tear, a remover is provided. Methods of applying the cosmetics, and kits containing the cosmetics, including a remover, are provided.

This patent application is a continuation-in-part of U.S. patent application Ser. No. 09/932,314 which is a continuation of U.S. patent application Ser. Number 09/665,885 filed Sep. 20, 2000, which was a continuation of U.S. patent application Ser. Number 09/472,355 (now U.S. Pat. No. 6,203,809), which was a continuation of U.S. patent application Ser. No. 09/266,469, filed Mar. 11, 1999, now U.S. Patent Number 6,027,739, which was a divisional of U.S. patent application Ser. No. 08/647,837, filed May 15, 1996, now U.S. Pat. No. 6,001,374, which was a continuation-in-part of US. patent application Ser. No. 08/440,780, filed May 15, 1995, now U.S. Pat. No. 5,747,017.

### BACKGROUND OF THE INVENTION

The present invention relates to cosmetic compositions that provide film layers to the human skin and its appendages. The film layers include or enhance coloring of, for example, lips and hair including brows and lashes. It is generally in a liquid form but may be modified to provide a balm with or without colors. Cosmetic compositions of the present invention include lip coloring compositions with or without enhancing agents, and formulations for removing the color. The present invention also relates to methods of using the compositions, and kits containing a plurality of the compositions.

Cosmetics have been a part of man's history. Coloring, enhancing, and conditioning hair, skin and nails have been goals through the ages. But in general, discoveries in cosmetics have been disjointed and without direction or general plans.

For example, lipstick has been utilized as a cosmetic preparation for heightening or altering the color of lips. Conventional lipstick is formed by a cosmetic coloring in a wax carrier. Although lipstick has many disadvantages, there has thus far been no suitable alternatives.

The lipsticks which have heretofore been available have the marked disadvantage of being readily transferable from a person's lips to other objects. Lipstick smears and rubs off while swimming, smoking, kissing, or by any other contact of the lips with articles such as coffee cups, tea cups, napkins and clothing. This leads to the loss of the lipstick application when drinking beverages, when swimming, and even when in engaging in none of these activities due simply to licking the lips. Thus, while lipstick is normally initially applies in a relatively even application across the externally exposed areas of the lips, the application of lipstick will readily dissipate and assumes a nonuniform coverage. The greatest loss of the lipstick application is typically near the portions of the lips closest to a person's mouth. As a consequence, it is necessary to frequently reapply layers of lipstick in order to maintain a uniform coverage of a lipstick application on a person's lips. This frequent necessity for reapplication aggravates a further disadvantage of conventional lipstick.

Lipstick has the additional disadvantage of being susceptible to cracking and caking. This disadvantage is particularly pronounced when several layers of lipstick have been applied to attempt to replenish a lipstick application that has been lost due to transfer to other articles. As a consequence, the frequent reapplication of lipstick results in perceptible cracking and caking of the lipstick covering a person's lips. Caking and cracking of a lipstick application detracts from the natural appearance of the lips and is aesthetically undesirable. Use of shellacs and waxes as major ingredients of lip stick increases the risk of cracking.

Similar problems attend the coloring of hair such as eyebrows and eyelashes. With regard to nails, nail polishes and enamels do not generally smear, but products available are harsh, e.g. contain acetone.

### SUMMARY OF THE INVENTION

A basic cosmetic composition of the present invention includes a film forming agent that may be contacted to human skin and its appendages including lips, hair including eyebrows and lashes, and nails. The film forming agent is a resin that is soluble in an organic solvent and insoluble in water. It is preferably prepared for use in the invention by combination with an organic solvent. The basic cosmetic composition by itself can be spread on human skin and its appendages to protect it, condition it and effect a shine. The basic cosmetic composition can also be used to alter a liquid cosmetic for coloring the lips of the present invention. An aspect of this use of the composition is to lighten the lip color.

The present invention presents a unified approach to conditioning and enhancing or coloring human skin and appendages by using a basic cosmetic composition that is a film forming agent, with combined pigment, natural extracts, enhancing agents, and the like. Included are innovative alternatives to lipstick. An embodiment of a lip cosmetic of the present invention used to apply color to the lips does not take the form of a gooey stick, but rather is a unique, smear-proof and waterproof liquid that dries quickly to an extremely sheer, soft finish that feels remarkably like bare skin. The lip cosmetic also is formed into a balm. In one embodiment, the resin of the present invention is present at 0.05-50% (preferably˜6%) the organic solvent at 30-90%.

In another embodiment, a lip coloring cosmetic includes at least 1% of a resin, for example, between about 0.1 and about 20 parts of a film forming resin, that is soluble in an organic solvent but insoluble in water, optionally between about 0.1 and about 15 parts of a cellulose, between about 0.1 and about 15 parts of a cosmetic pigment, and between about 50 and 99 parts of an organic solvent.

The alcohol soluble, water insoluble resin which is employed is the critical component that provides the cosmetic of the invention with its numerous advantages over lipstick. Although AMPHOMER is preferably utilized as the requisite alcohol soluble, water insoluble resin, other film-forming agents may be used not only to add a white component of color, but also to serve as a sunscreen.

If the basic cosmetic is to be used on facial skin, oils, triglyceride, panthenol, propylene glycol or a mixture thereof, glyceryl esters and derivatives, panthenol, esters and fatty acids, fatty alcohols, unsaponifables or silicone may be combined up to ˜40% (preferred up to ˜5%). Ethylcellulose is optionally added.

A composition of the film-forming agent is useful to apply to hair. The composition aids hair setting and quick drying. It can be combined with pigment.

The present invention also relates to compositions for enhancing the finish of the cosmetic. An enhancing agent that produces a gloss or shine when contacted to the skin or its appendages is another aspect of the present invention. The enhancing agent also preferably has a moisturizing effect. When used with the lip coloring agent of the present invention, the enhancing agent acts as a base coat and a conditioner. The enhancing agent may be clear or may include a tint. The tinted version offers a variation in lip coloring when used with the lip coloring cosmetic.

The enhancing agent used alone will wear off. It does not have to be removed with the remover solution. To prevent the enhancer from wearing off it must be “locked” in with the liquid lip coloring agent or the colorless film forming composition. These two compositions provide a resin as disclosed herein.

A multi-purpose, all weather, protective moisturizing lip balm can be used alone or as a base conditioner when used as a coating for the liquid lip cosmetic of the present invention. The lip balm is waxless. A lip balm is prepared by adding a solidifying agent such as silica to a formulation for an enhancing agent.

Such compositions, when applied over the cosmetic, transform the finish of the cosmetic from a matte to a high-gloss finish. The finish enhancing compositions also keep the lips soft and moist and may be massaged lightly into the lips prior to application of the cosmetic to condition and moisturize the lips. The finish enhancing compositions may be applied with an applicator wand. The user can apply the finish enhancing composition multiple times, whenever it is desired to moisturize the lips or to enhance the finish of the cosmetic.

The present invention also relates to compositions for removing the cosmetic, finish enhancer, and color enhancers. These compositions may be dabbed on a tissue and applied to the lips when the user desires to remove the preceding compositions from the lips. Preferably, the remover is applied before applying the cosmetic compositions to prepare the surface for optimum adherence.

The present invention also relates to kits to provide a plurality of the compositions of the present invention and to methods of using them, that is, of applying them to skin and its appendages.

Kits are combinations of compositions of the present invention that are packaged and sold as a unit, or advertised as a unit. The unit may either be a single package such as a box or bag, or compartments linked together for purchase, or recommended for purchase in a unit in other ways, for example, by advertising them as a unit or offering a unit price discount. The unifying theme of the present invention is to provide smear-resistant products that stay on the human epidermis longer than previously reported products. The improvement is due to use of a film-forming agent that preferably is a resin that is soluble in an organic solvent such as alcohol, but insoluble in water. Because of the properties of the film-forming agent, a remover specifically designed to remove it and whatever other agents are applied to the skin and its appendages with the film-forming agent is offered either as part of the kit or as a recommended purchase.

In the kit, the source of the film-forming agent, the agents used in conjunction with it, and the remover may be in separate compartments. The compartments may be independently structured, for example as separate vials in box, or linked such as components of a single complex such as parts of a tube. Some of the sources, except for the remover, may be combined, for example the film forming agent and the pigment. The remover would not e combined with the other ingredients in the same compartment because the remover, by definition, negates the film forming characteristics of the other components of the kit. The remover may be provided in a foam by means of adding gas.

Some of the kit components may be provided in a towelette form, e.g. the remover, the enhancing agent.

Embodiments of a kit include:

1. a film forming agent

a pigment for coloring lips

a colorless enhancing agent

a remover

2. a film-forming agent

a pigment for coloring lips

a tinted enhancing agent

a remover

3. components of 1 or 2 where the pigment is for coloring hair, eyebrows or eyelashes

4. a colorless lip balm

a film-forming agent

a pigment (preferably combined with the film forming agent)

a remover

5. 4 with a tinted lip balm instead of a colorless one

6. lip balm (colorless or tinted)

a remover

7. film-forming agent

8. color for eyes

enhancing agent for eyebrows and lashes

a remover.

The cosmetics of the present invention are both smear-proof and waterproof.

They will not streak, smear, or rub off while swimming, smoking, or kissing. Use of the cosmetics of the present invention avoids the disadvantages of stains on coffee cups, cheeks and collars. The cosmetics of the present invention have twice the staying power of analogous cosmetics such as lipsticks or balms which purport to be waterproof or kiss-proof.

The cosmetics of the present invention have further advantages in that they won't stick to dental work or braces. They can be used on hair, such as lashes or eyebrows, as well as to cover any bald areas on the head. It can be used to cover scars or tattoos anywhere on the body.

The present invention provides a perfect cosmetic for busy professional women. It is highly advantageous for wear at weddings, parties, for use at the beach, for use while skiing and for wear during all active sports. The cosmetic of the invention is extremely attractive and can be worn at important dinner dates, as well as during informal events. The wearer can even sleep or shower while wearing the cosmetic, since it will not fade or smear under such circumstances. The user may apply several layers of the cosmetic to achieve the desired appearance.

All of the liquid formulations of the cosmetic with film-forming agent of the foregoing examples will dry in a thin film, when applied to the lips. The cosmetic will not crack or cake eve with repeated applications. When any of the foregoing formulations are applied to a wearer's lips in at least three layers, the cosmetic covering provided will last a wearer engaged in virtually any normal activity throughout an entire day without fading or rubbing off.

The formulations disclosed herein may also be used to apply color to skin, nails, hair or to cover bald spots. In addition, the above compositions can be used as liners to provide definition. For use as a liner, darker pigments are preferred, although any pigment will work. Additionally, if used as a liner, the composition should be applied to the lips with a fine tip brush.

The cosmetic of the invention may also employ vitamins, minerals, antioxidants, drugs, organic compounds, herbs, proteins, plant extracts, root extracts, enzymes, sorbitol, pectin and PCA to take advantage of their known coloring, flavoring, moistening and adhering properties.

Additional components may be added to the above composition including flavoring agents, skin conditioning agents, emollients, skin protectants, sunscreens, UV high absorbers, anti-oxidants, humectants, essential oils, minerals, PABA, heterocyclic compounds, oils, fats, and fatty acids.

Representative flavoring agents which may be used in the present compositions include cinnamon, peppermint extract, saccharin, Acesulfame K and other flavoring agents such as those listed in the CTFA Cosmetic Ingredient Handbook 2d. ed., published by the cosmetic, Toiletry, and Fragrance Association, 1101 17^{th} St. N.W., Suite 3000, Washington, D.C. 20036 (1992). The amounts and identities of such flavoring agents may be adjusted to provide a desirable flavor to the composition.

Numerous skin conditioning agents may be selected for use in the present compositions, provided they are oil soluble. These include the emollients, humectants, miscellaneous, and occlusive skin conditioning agents listed in the CTFA Cosmetic Ingredient Handbook. The amounts and identities of such skin conditioning agents can be adjusted to provide the desired results. A highly preferred humectant is glycerin. Many UV absorbing compounds are known to those skilled in the art, including those listed in the CTFA Cosmetic Ingredient Handbook. However, the preferred UV absorber is octylcrylene.

Numerous sunscreen agents are known to those skilled in the art, including those listed in the CTFA Cosmetic Ingredient Handbook. However, a preferred sunscreen is octyl methoxycinnamate. In a preferred version of this aspect, the antioxidant is a vitamin E linoleate mixture available from Seltzer Chemicals, Carlsbad, Calif.

Many skin protectants are known to those skilled in the art, including those listed in the CTFA Cosmetic Ingredient Handbook.

Many essential oils, minerals, PABA, heterocyclic compounds, oils, fats, and fatty acids suitable for use in the present compositions are known to those skilled in the art. Representative compounds in each of these categories are listed in the CTFA Cosmetic Ingredients Handbook. Effective amounts of such compounds may be included in the present compositions to achieve the desired effect.

DEFINITIONS

Skin and Its Appendages—this term as used herein includes epidermal layers, lips, facial skin, nails, hairs, cuticles (See Gray's Anatomy).

Hairs—include eyelashes, eyebrows.

### DETAILED DESCRIPTION OF THE INVENTION

The cosmetic of the present invention includes a basic cosmetic composition that includes a film-forming agent, preferably prepared in an organic solvent; a lip coloring cosmetic which is the basic cosmetic with pigment; an enhancing agent; a treated enhancing agent; a waxless lip balm with or without tint; hair and skin coloring agents; and removers.

The cosmetics of the invention contains no petroleum products. Small amounts of waxes are optional, as needed for pigment dispersions. Waxes can smear while petroleum products can burn or dry the skin.

Lip Coloring Cosmetic

The basic cosmetic composition of the invention is so sheer that a wearer can layer on at least three different layers without any danger of the product caking up, for example, on the lips. Indeed, the different layers of the cosmetic of the invention can be applied in different colors to achieve a unique, aesthetic effect.

Also, a different number of layers of the cosmetic can be applied to produce different aesthetic appearances. A single layer basic cosmetic of the invention dries to a shear finish. If a second layer of the cosmetic is applied over the first, a medium matte finish is achieved. By applying a third layer, a wearer achieves a full cover matte finish. Even with use of multiple layers, however, the basic cosmetic of the invention will not cake or smear.

The basic cosmetic of the invention can be formulated in any number of different colors by varying the color of the pigment employed. The basic cosmetic of the invention may be applied in a liquid form and the colors can be used individually or mixed by layering to create an unlimited array of custom, personalized colors. Each layer takes about twenty seconds to dry from the time of application.

In addition to its basic components, the cosmetic of the invention may include other substances to achieve certain effects. For example, the basic cosmetic may be formulated with at least about one part of a dimethicone component to achieve a gloss or satin effect. Although the cosmetic formulated in this manner looks and feels viscid or sticky, it will not come off, but will stay on the lips despite extended wear and exposure to moisture.

The basic cosmetic may also be formulated as a lip liner by utilizing a higher concentration of cosmetic dyes or pigments in the formulation. When formulated in this manner, the cosmetic may be utilized to outline the lips in the same or a different color as the basic application.

The basic cosmetic of the invention provides natural sun protection and keeps the lips from chapping, indoors, or outdoors, in both cold and hot weather. It will not stick to teeth or dental braces. Unlike a user wearing lipstick, an individual wearing the cosmetic of the present invention can actually brush and floss after means without smearing the cosmetic or reducing the thickness of its lip coating, and without having to reapply the lip covering.

The cosmetic of the invention is organic and hypoallergenic. Unless a flavor or fragrance is added, it is also odor free and contains no petroleum products.

The lip cosmetic of the invention enhances the beauty of the wearer without surgery by filling in the inner lips where conventional lipstick will not stay. The lip cosmetic of the invention is extremely sheer and is not gooey like conventional lipstick. Even after three or more layers of the cosmetic of the invention are applied, the lips still fee bare and have a perfect matte finish.

The liquid lip cosmetic of the invention is not at all viscous, and can even be poured from a container. Its sheer consistency allows it to be applied with a fine tip applicator so that it can be applied with the precision of a makeup artist.

The cosmetic of the invention will not come off with petroleum jelly or cleansing cream. Due to its permanence, care must be taken not to spill the cosmetic of the invention in liquid form onto clothing or other fabrics. However, if the cosmetic is accidentally spilled onto carpeting or clothing, it can be readily removed by applying water and then applying isopropyl alcohol or the cosmetic removing formulations described below, provided that cleanup is undertaken promptly.

To use the lip cosmetic of the invention, it is recommended that the lip area be cleaned thoroughly with the cosmetic removing compositions described below. A bottle of the cosmetic containing small mixing balls should be shaken for four or five times until the mixing balls move freely within the bottle.

The lip cosmetic of the invention is then applied generously in liquid form to dry, clean lips. It is recommended that three consecutive layers be applied at a time for full day-time coverage. The cosmetic can be applied with a soft doefoot applicator or brush applicator and should be applied across the lips in a single direction only. When applying the cosmetic, the wearer should keep in mind the freedom of creating fuller lips by filing in the inner lips where regular lipstick does not stay. Approximately 20 seconds should be allowed to elapse between consecutive coats in order to allow the immediately preceding coat to dry. During this time, the lips should be kept apart and not blotted. For best results after the application of the cosmetic of the invention, the wearer should refrain from eating, drinking or smoking for at least one minute.

With the application of the cosmetic, the wearer's lips may tingle at first. This tingling sensation diminishes or goes away entirely with repeated use, because the sealing effect of the product actually helps to eliminate the mild, but ever present chapping common to most lips.

The tingling sensation which is sometimes present may be avoided by applying at first a thin layer of the cosmetic containing dimethicone. Subsequent layers of the same or a different formulation of the cosmetic may be applied to achieve the desired degree of sheer or matte finish.

The bottle containing the cosmetic of the invention should be kept closed after each use. With daily use, a one quarter bottle should last approximately two months.

Film-Forming Agents

An improvement of the invention is use of at least one film-forming agent present in an amount of at least about one percent wherein the film-forming agent is selected from the group consisting of: AMPHOMER (copolymers of N-tert-octylacrylamide, methyl methacrylate, hydroxypropyl methacrylate, acrylic acid and/or t-butyl aminoethylmethacrylate), AMPHOMER LOVOCRYL-47 (an octylacrylamide/acrylate/butylaminoethyl methacrylate copolymer), Carboset, JONCRYL (styrene/acrylate copolymers), QUADAMER (terpolymers of alkyl acrylamide, acrylamide or methacrylamide, N-vinyl pyrrolidone, and acrylic or methacrylic acid), GANTREZ (copolymers of vinyl methyl ether and monoalkyl esters of maleic anhydride), and polyvinyl acetate copolymers.

Five performance tests were conducted on a variety of resins to determine their suitability for use in the present cosmetics. The suspension test was employed to assess the ability of the test compound to be solubilized in a solution of 4.4% test resin, 3.5% ethyl cellulose, 0.5% castor oil, 0.8% D&C Red #7 Calcium Lake pigment, and 90.7% ethyl alcohol.

The rub test was deigned to determine the test resin's ability to be retained on the skin. The composition used for the suspension test was painted on the skin and allowed to dry. Thereafter, the skin was rubbed under cool tap water until the composition began to fall apart. A composition received a score of “Good” if it was able to withstand five minutes or more of rubbing. A compound received a score of fair or poor if it withstood less than one minute of rubbing.

In the color test, the color of the resin containing compositions used in the suspension test was applied to the lips and the quality of the color obtained was compared to that produced using a composition lacking the test resin.

In the “feel on the lips” test, the compositions used in the suspension test the compounds were evaluated to determined whether they produced a tight shrinking feeling, cracked, dried the lips, or caked on the lips. Compounds performing favorably in this test produced none of the preceding effects and were not noticeable to the wearer.

In the drying time test, the compositions used in the suspension test were applied to the lips and the length of time the composition took to dry was evaluated.

Preferentially the compositions take about 20 seconds to dry.

The above tests were performed on the following resins:

(1) Resin 28-2930 (VA/coronates/vinyl neodecanoate copolymer) available from National Starch, Bridgewater, N.J.

(2) Amphomer LV-71 (octylacrylamide/acrylates/butylamino ethyl methacrylate copolymer) available from National Starch.

(3) Water Lock G40-A 180-D242 (corn starch/acrylamide/sodium acrylate copolymer) available from Grain Processing Corp., Musscatine, Iowa 52761.

(4) Daihold (amp/acrylate copolymer) available from Sandoz Chemical Corp., Charlotte, N.C.

(5) Eastman AQ-385 and AQ-555 (Diglycol/cyclohexanedimethanol/isophthalates/sulfylisophthates copolymer) available from Eastman Kodak, Rochester, N.Y.

(6) Ultra Hold 8 (acrylates/acrylamide copolymer) available from Base Corp., Clifton, N.J.

(7) Omnirez 2000 (2 butenedioic acid 2-monoethyl ester polymer with methoxyethene available from JSP, Sherman Oaks, Calif.

(8) Gantarex compounds such as A-425, ES 425, and ES-435 (which are butyl esters of PVM/MA copolymers), ES-335 (isopropyl ester of PVM/MA copolymer), or ES-225 and SP-215 (esthylesters of PVM/MA copolymer), all of which are available from ISP, Sherman Oaks, Calif.

(9) H2old EP-1 Terpolymer (Vinyl caprolactam/PVP/diethylaminoethyl/methacrylate copolymer) available from ISP, Sherman Oaks, Calif.

(10) Amphomer Lovocryl-47 (octylacrylamide/acrylates/butylaminoethyl methacrylate copolymer) available from National Starch, Bridgewater, N.J.

(11) Amphomer 28-4910 (octylacryl amide/acrylates/butylamino ethyl methacrylate copolymer) available from National Starch, Bridgewater, N.J.

(12) Advantage Plus terpolymer (VA/butylmaleate/isobornyl acrylate copolymer) available from ISP, Sherman Oaks, Calif.

(13) Copolymer 958 (PVP Dimethyl amino ethylmethacrylate copolymer) available from ISP, Sherman Oaks, Calif.

(14) Joncryl (styrene/acrylates copolymer) available from SC Johnson Polymer, Racine Wis.

(15) Sentry Polyvinyl acetate-12 (polyvinyl acetate) available from Union Carbide Corp., Houston, Tex.

(16) Carboset-525 (acrylates copolymer) available for B.F. Goodrich, Brecksville, Ohio.

Table I summarizes the results of the performance tests.

Resin 28-2930, Amphomer LV-71, Amphomer Lovocryl-47, and Amphomer 28-4910 received an overall performance rating of “good” in the above performance tests. While any of these substances tested in the performance tests could be utilized in the formulation of the cosmetic of the invention, the alcohol soluble, water insoluble resin that serves as the film-forming agent preferably includes an AMPHOMER component. The most highly preferred AMPHOMER is Amphomer LV-71. In the preferred formulation the film-forming agent is preferably comprised of an AMPHOMER and ethyl cellulose.

AMPHOMER is a trademark under which copolymers of N-tert-octylacrylamide, methyl methacrylate, hydroxypropyl methacrylate, acrylic acid and t-butyl aminoethyl methacrylate are sold. GANTREZ is a trademark under which copolymers of vinyl ethyl ether and mon-alkyl esters of maleic anhydride are sold by GAF. QUADMER is a trademark under which teropolymers of alkyl acrylamide, acrylamide or methacrylamide, N=vinyl pyrrolidone and acrylic or methacrylic acid are sold by American Cyanamid. The most preferred film-forming agent is the commercially available AMPHOMER sold as LV-71 by National Starch and Chemical Company, Specialty Polymers, having an address of 10 Finderne Avenue, P.O. Box 6500, Bridgewater, N.J. 08007-3300.

The amphoteric acrylic resin forming the chemicals sold in the trade as AMPHOMER has previously been utilized as a fixture in hair spray as described, for example, in U.S. Pat. Nos. 4,192 and 4,315,910. However, Applicant has discovered that this substance can also serve as the preferred form of the critical ingredient of a cosmetic which forms a clear, colorless film on the lops and which has a staying power far greater than that of conventional lipsticks.

The AMPHOMER utilized in the cosmetic of the invention functions as a transparent sealer. It binds and seals the cosmetic to the lips in a clear, sheer film which does not crack or cake. The film-forming agent in the cosmetic composition is what makes the product so long lasting.

The AMPHOMER is an amphoteric acrylic resin. Its official Cosmetic Toiletries and Fragrances Association (CTFA) product designation is Octylacrylamide/Acrylates/Butylaminoethyl Methacrylate Copolymer. AMPHOMER is carboxylated at regular intervals along its molecular chain. In its undissolved form it is a fine white free-flowing powder. It has an intrinsic viscosity of 0.40 in ethanol at 25 degrees Centigrade. It contains about three percent volatiles and has an acidity of 2.05 me/gram.

In one embodiment, the invention may be considered to be a cosmetic comprising between about 0.1 and about 20 parts of a resin soluble in alcohol and insoluble in water, between about 0.1 and about 15 parts ethyl cellulose, between about 0.1 and about 15 parts of a cosmetic pigments, and between about 50 and 99 parts of an organic solvent. All parts used herein are percent by weight.

Preferentially, the solvent is denatured alcohol. In an embodiment, the alcohol soluble and water insoluble resin is present in between about one and about 10 parts. Ethylcellulose is optional. If used, the ethylcellulose is preferably in between about 1 and about 10 parts. In an embodiment, the cosmetic pigment is present in between about 2 and about 10 parts.

The AMPHOMER utilized in the cosmetic of the invention functions as a transparent sealer. It binds and seals the cosmetic to the lips in a clear, sheer film which does not crack or cake. The film-forming agent in the cosmetic composition is what makes the cosmetics long lasting.

Organic Solvent

The organic solvent or carrier employed is preferably denatured alcohol, sometimes termed ethyl alcohol or ethanol. Other organic solvents which may be employed instead of or in addition to denatured alcohol include stearyl alcohol, cetyl alcohol, cetearyl-cetostearyl alcohol, SDA alcohol, methyl alcohol, isopropyl alcohol and C18-40 alcohol. Other organic solvents which may be employed include higher fatty-acids which are immiscible in water. These include lauric acid, myristic acid, stearic acid, palmitic acid, behenic acid, and lanolin fatty acid. Lanolin and triterpene may also be employed in the organic solvent.

Depending upon the concentration of the other components, the organic solvent, which is preferably SDA 40B 190 denatured alcohol, is preferably present to the extent of between about 30 to 95 percent. Within this range, a concentration of 80 to 95 percent organic solvent may be utilized.

Pigments

The pigments which are employed to provide coloring to the cosmetics are normally provided as dispersions in castor oil. The degree of concentration of the pigments in castor oil determines the shade of the coloring. Normally a concentration of between 25-65 percent pigments in castor oil acts like a plasticizer and also makes the film formed more flexible.

The pigment and castor oil are preferably present in the cosmetic of the invention in an aggregate amount of between about 0.1 percent and about ten percent. Alternatively, the pigments may be alcohol soluble pigments, such as D&C Red 28 Lake, in which case a castor oil dispersion is not necessary to solublize the pigments.

Pigments of this type are standardized in the cosmetic industry and are identified in that industry by FD&C designations, D&C designations, and natural color designations that are compatible with alcohol solutions. The preferred pigments that are employed include D&C Red No. 6, Barium Lake, D&C Red No. 7, Calcium Lake, D&C Orange No. 5, D&C Red No. 27, FD&C Yellow No. 5, FD&C Blue No. 1, iron oxide and others. The D&C lake colors are all made with iron, aluminum, calcium, barium, potassium, strontium or zirconium.

Other pigments which may be employed include those of dye or coal tar origin and chemical compounds used as pigments. Inorganic colors such as iron oxides may be utilized. White pigments may be formed of titanium dioxide, zinc oxide, mica or pearls. Pigments formed of nitro dyes may be utilized if desired. These dyes contain one atom of nitrogen and two of oxygen. However, only a few nitro dyes are certified by the FD&C or D&C because nitro dyes can be absorbed through the skin and some are toxic. However, D&C Yellow No. 5 is one acceptable nitro dye which can be used as a component of the pigment of the invention.

Azo pigments may also be utilized. These pigments are characterized by the presence of the azo bond, and monoazo pigments include the greatest number of pigments within this group.

Another group of suitable pigments is the triphenylmethane group. FD&C Blue No. 1 is the most popular dye of the group and is widely used in the cosmetic industry. Xanthene pigments may also be utilized. This group of pigments includes Berry brilliant which is widely used in lipstick colors, as well as D&C orange.

Certain of the quinoline dyes are also suitable for use as pigments. There are only two certified cosmetic colors in this category, namely D&C Yellow Nos. 10 and 11. These are bright greenish yellow colors.

The anthraquinone dyes are also suitable for use in formulating the pigment required by the compositions of the invention. These dyes are widely used in cosmetics because they are not affected by light. D&C Violet No. 2 is one example of such a dye suitable for use in the invention. The anthraquinone dyes should not be used in the lip area, but may be used in other applications, such as on the nails.

Indigo dyes are also suitable for use and have been used in cosmetics for may years. D&C Blue No. 6 is one example of a suitable indigo dye.

Pigments of vegetable, animal, or mineral origin may also be utilized according to the invention, One suitable pigment of animal origin is cochineal extract. Natural colors and vegetable compound pigments which may be utilized include alkanet, annatto, carotene, chlorophyll, saffron and tumeric, beet juice powder, carmine alkanet root, carmel, grape skin extract, and beta carotene. Hydroxyascetone and indelible dyes may also be utilized in formulating the pigment in the cosmetic of the invention.

Aluminum Lake Yellow Nos. 5, 6, 10; Zirconium Lake Yellow No. 5; Yellow Nos. 5, 6, 10; Aluminum Lake Red Nos. 21, 22, 27, 28, 30, 33, 40; Red Nos. 6, 21, 22, 27 28, 33, 36; Zirconium Lake Red Nos. 21, 27, 33; Potassium Lake Red No. 6; Varium Lake Red No. 6; Sodium Salt Red No. 6; Calcium Lake Red No. 7; Talc Lake Red No. 30; Aluminum Lake Blue No. 1; Zirconium Lake Orange No. 5; Aluminum Lake Orange No. 5; Orange No. 5; Green Nos. 3, 5.

The pigments may contain 0.1 to 5% stain. Preferably the pigment contains 0.65%-1.5% stain.

Enhancing Agents

The present invention also contemplates compositions for enhancing the finish of the cosmetic. An enhancing agent that produces a gloss or shine when contacted the skin and its appendages is another aspect of the present invention. The enhancing agent also has a moisturizing effect. When used with the lip coloring agent of the present invention, the enhancing agent acts as a base coat and a conditioner. The enhancing agent in combination may be clear or may include a tint. The tinted version offers a variation in lip coloring when used with the lip coloring cosmetic.

The enhancing agent used alone will wear off. It does not have to be removed with the remover solution. To prevent the enhancer from wearing off it must be “locked” in with the liquid lip coloring agent or the film forming composition. These two compositions provide a resin as disclosed herein.

The finish enhancing agent can be applied over the basic cosmetic to enhance the matte finish of the cosmetic alone into a high-gloss finish. In addition, the lips remain soft and silky while leaving the waterproof and smearproof cosmetic in place. The finish enhancing compositions may also be massaged lightly into the lips prior to application of the cosmetic to condition and moisturizing the lips.

The finish enhancing composition may include a silicone. The silicone may be a silicone copolymer. In one version of this composition, the composition comprises about 100% silicone. The silicones function to provide a water barrier, gloss and spreading and wetting activity. They may also include moisturizing abilities and may function as a carrier for other active ingredients such as sunscreens or vitamins. Mixtures of different silicones may also be used to achieve the desired moisturizing, carrier, or other beneficial effects. Virtually any of the silicones offered by GE Silicones will work in the present invention, including Cyclomethicone, Dimethicone, mixtures of the Cyclomethicone and Dimethicone, Dimethicone and Laureth-4 and Laureth-23, Dimethicone Copolyol, Cyclomethicone and Dimethicone Copolyol, Trimethylsilylamodimethicone, and other silicones provided by GE Silicones for use in personal products. Preferentially, the silicone is a silicone copolymer. Preferred silicones are the Dimethicones available from GE Silicone and dimethylsiloxane methyl (polyoxytheylene) siloxane copolymer, such as the Dimethicone and Trimethylsiloxane available from GE Silicones. Representative dimethicones include a polydimethylsiloxane have a viscosity of 5 centistokes at 25° C. such as SF96® (5), a polydimethylsiloxane having a viscosity of 20 centistokes at 25° C. such as SF96® (20), a polydimethylsiloxane having a viscosity of between 50 and 1000 centistokes at 25° C. such as SF96® (50-1000), a polydimethylsiloxane having a viscosity of 60,000 centistokes at 25° C. such as Viscasil® 60M, a blend of 15% of a high molecular weight methyl terminated polydimethylsiloxane fluid gum having a penetration between 500 and 1500 mm and 85% of a polydimethylsiloxane which has a viscosity of 5 centistokes at 25° C. such as SF1236, and a high molecular weight methyl terminated polydimethylsiloxane fluid gum having a penetration between 500 and 1500 mm such as SE30 all of which are available from GE Silicones.

In another version of this embodiment, the finish enhancing composition comprises a silicone, a lipophilic gelling gent and a preservative. The silicones suitable for use in this embodiment are the same as those for the composition above. The lipophilic gelling agent acts as a carrier for introducing additional components into the composition. Desirable additional components are discussed below. In a highly preferred embodiment, the lipophilic gelling agent is a cyclomethicone pentamer and aluminum magnesium hydroxide stearate such as Gilugel SIL-5 (produced by Giulini Chemie, Germany and available from Morse Chemical, Inc., San Gabriel, Calif.). A preferred preservative is phenoxyethanol. In a highly preferred version of the present invention, the finish enhancing composition comprises about 1 to about 99% silicone, about 0.1 to about 50% lipophilic gelling agent, and about 0.1 to about 10% preservative. In a highly preferred version the dimethylsiloxane methyl (polyoxytheylene) siloxane copolymer is present at a concentration of 88.40%. In a highly preferred version, the Gilugel SIL-5 is present at 10%. Preferentially, the preservative is phenoxyethanol or BHT (butylated hydroxytoluene). In a preferred version, the phenoxyethanol is present at 1.6%. In another preferred embodiment, the BHT is present at 1.6%.

Instead of silicone, triglycerides such as shea butter, castor oil, oils or fats such as cholesterol may be substituted, at least in part.

The enhancing agents are part of the a waxless balm and can be used with the lip balm. They are compatible and they can be used together, especially mixing clear enhancer with a tinted waxless lip balm. They mix and match.

Remover

The present invention also discloses compositions for removing the cosmetic. One version of the cosmetic remover is a solution comprising a mild detergent plus a preservative. Preferentially, the mild detergent is present from between about 0.5 parts and about 10 parts and the preservative is present between about 0.1 and about 3 parts. A preferred mild detergent is sodium lauryl sulfate and a preferred preservative is Quaternium 15 (Dowicil 200 available from Dow Chemical). In a highly preferred embodiment of this composition, the sodium lauryl sulfate is present at 0.5 parts and the preservative is quaternium 15 (Dowicil 200 available from Dow Chemical) at 0.1 parts, with the remainder of the composition being water.

Alternatively, the lip removing composition may comprise a solution of alcohol and a chelating agent. In a preferred version of this embodiment, the alcohol is ethyl alcohol and the chelating agent is trisodium phosphate. Preferentially, the trionum phosphate is present at about 0.1-85 parts by weight.

In yet another embodiment, the cosmetic removing composition comprises an aqueous solution of a chelating agent, one or more mild detergents, and a preservative. Preferentially, the water is distilled. Preferentially, the chelating agent is trisodium phosphate. Preferred mild detergents are Empigen CDR 30 (cocoampho acetate available from Albright & Wilson, Cumbria, United Kingdom) and the nonionic surfactant polyoxytheylene polyoxypropylene glycol (Pluronic® F127, Poloxamer 407). Preferred preservatives are phenoxyethanol, sodium benzoate, and Quaternium 15) (Dowicil 200 available from Dow Chemicals). In further aspects of this embodiment, one or more thickeners are added. A preferred thickener is xanthan gum. The xanthan gum also functions to maintain the solubility of sodium lauryl sulfate and trisodium phosphate in alcohol based formulations. In yet further embodiments of the cosmetic removing composition a flavoring may be added. Preferred flavorings are Acesulfame K (a sweetener Sunnett Brand sweetener available from Hoechst Celanes, 3340 W. Norfolk Rd., Portsmouth, Va. 23703), and sodium saccharin.

Preferentially, the chelating agent is present between about 0.5 and about 5 parts. It is preferred that the detergents be present from about 5.05 to about 20.5 parts. Preferentially the preservative is present between 0.1 and 5 parts. Preferentially, the thickener is present between about 0.05 and 10 parts. Preferentially the sweetener is present between about 0.05 and 5 parts.

Mild alkali solutions may also be used to remove the cosmetic. A preferred aspect of this embodiment is 0.5-5 parts sodium borate, with the remainder being water. In a highly preferred embodiment, the sodium borate comprises 1.78 parts of the aqueous solution.

The remover formulations in the examples can be used for skin, lips, eyebrows and lashes, and nails.

The removers of the present invention are not necessary for removal of the enhancing agent alone. The remover can be prepared in either a liquid form or a foaming form. The foaming form is dispensed through a pressure valve on the product dispenser.

Color Enhancing Compositions

The present invention also relates to compositions for further enhancing the color of the cosmetic. These color enhancing compositions comprise colorants such as mica, bismuth oxychloride, iron oxides, D&C Lake colorants, FD&C Lake colorants, D&C colorants, and FD&C colorants. The color enhancing compositions are provided in pressed or loose form. If provided in pressed form, the colorants are mixed with preservatives. Preferred preservatives are ethyl paraben, methyl paraben, or polyparaben. Additionally, if provided in the pressed form, the color enhancing composition also comprises a wax such as corn glutin protein or a synthetic wax and a C12-C15 alkyl benzoate.

The invention may be further illustrated by way of the following examples.

### EXAMPLES

Example 1: Preparation of a Film-Forming Composition

The first step in formulating a film-forming composition (basic) of the invention is to create a sealer. The sealer is formed by mixing a film-forming agent in an organic solvent. Specifically, suitable film-forming agents include 4.4 parts of Amphomer LV-71, obtained from National Starch and Chemical Company, and 1.9 parts Etocel -22, obtained form Aqualon Corporation, are mixed at room temperature in 93.7 parts 190-proof specially denatured alcohol, sold as SDA 38B-190. The Ethocel -22 provides ethyl cellulose which functions as an adjacent film former in the sealer.

Example 2: A Colored Film-Forming Composition

A. A sealer is first prepared by mixing, for example, 4.3 parts by weight of Amphomer LV-71 along with 3.2 parts by weight Ethocel N-4 into 92.5 parts specially denatured alcohol SDA 40-B-190. 8.25 parts of D&C Red No. 7 pigment dispersion in castor oil along with 0.25 parts D&C Orange No. 5 pigment dispersion in castor oil along with 0.25 parts D&C Orange No. 5 pigment dispersion in castor oil are then mixed with an additional 0.5 parts castor oil and introduced into the quantity of sealer previously prepared. The sealer, therefore, constitutes 90.75 parts by weight of the total composition. The pigment grinds and additional castor oil are stirred into the sealer to produce the finished liquid cosmetic composition according to the invention.

Five parts by weight of a dispersion of D&C Red No. 7 Calcium Lake in castor oil and 0.5 parts by weight of a dispersion of D&C Orange No. 5 in castor oil are then selected for use as the pigment or coloring agent. Both the D&C Red No. 7 Calcium Lake and the D&C Orange No. 5 each comprise about 50 percent of the total weight of their respective dispersions. The remaining weight of the dispersions is attributable to the castor oil. The pigments should be obtained in as finely ground a form as possible. The pigment dispersions are mixed together along with another 0.5 parts by weight castor oil.

The pigment and castor oil mixture is then mixed in with the sealer, also at room temperature. The AMPHOMER, the Ethocel and the pigment ingredients are all readily soluble in the denatured alcohol. When mixed, the formulation forms a somewhat turbid solution. Pigment grinds can be stirred into the sealer in any convenient manner. The resultant liquid mixture is one preferred embodiment of a film-forming cosmetic according to the invention.

B. The organic solvent can be from 45% to 98% the Amphomer can be from 0.01-15% other additives can be added such as up to (10% water), the denatured 190 proof ethyl alcohol has 3% water in it with a 7% denaturant of Rosemary oil. Other additives can be Shellac (de waxed bleached), oils and triglycerides, silicones, cellulose, silica, thickeners etc, vitamins, minerals, fragrance, etc. The additives can be used to reduce the alcohol as well as provide a smell or a texture or to create a more pliable feel. Increasing the alcohol can quick dry faster.

Example 3: A Film-Forming Composition With a Gloss

The formulation of Example 1 is repeated, but with the addition of two parts methyl silicone to the sealer prior to adding the pigments to the sealer. The use of a dimethicone such as methyl silicone increases the shininess of the cosmetic.

Example 4: A Moisturizing Film-Forming Composition (Gloss)

The formulation of Example 1 is repeated, but with the addition of two parts by weight of glycerin as a humectant in producing the sealer. The use of a humectant aids in moisturizing the lips.

Example 5: A Formulation for Applying Color Around the Eyes

Furthermore, with slight modifications, the above formulations may be used to apply color around the eyes. For application around the eyes, an aqueous solvent is used, the ethylcellulose is omitted, and Aminonathyl amino methyl propanol is added. A suitable composition for use around the eyes comprises between about 0.1 and about 20 parts of a water soluble resin, between about 0.1 and about 15 parts of a cosmetic pigment, between about 0.5 and about 4 parts Aminonathyl amino methyl propanol.

Preferred finish enhancing compositions are listed in Examples 6-10.

Example 6: Finish Enhancing Composition 1

| AMOUNT | |
|---|---|
| (BY WEIGHT) | COMPOUND |
| 88.40 | Dimethylsiloxane methyl (polyoxytheylene) |
| | siloxane copolymer |
| 10.00 | Gilugel SIL-5 |
| 1.60 | Phenoxyethanol |

To formulate the above composition, the Gilugel SIL-5 is heated until it melts. The phenoxyethanol is then added thereto, and the resulting mixture is added to the Dimethylsiloxane methyl (polyoxytheylene) siloxane copolymer.

Example 7: Finish Enhancing Composition 2

| AMOUNT | |
|---|---|
| (BY WEIGHT) | COMPOUND |
| 7.00 | Dimethylsiloxane methyl (polyoxytheylene) |
| | siloxane copolymer |
| 5.50 | Gilugel SIL-5 |
| 0.30 | BHT (butylated hydroxytoluene) |
| 1.00 | Phenoxyethanol |
| 84.10 | Dimethicone |
| 1.00 | Octylcrylene |
| 1.00 | Octyl methoxycinnamate |
| .10 | Vitamin E linoleate mixture |

The composition is prepared by heating the Gilugel to the melting point and adding the octylcrylene, octyl methoxycinnamate, vitamin E, phenoxyethanol and BHT. The two silicones are separately mixed and the Gilugel mixture is then added thereto.

Example 8: Finish Enhancing Composition 3

| AMOUNT (BY WEIGHT) | COMPOUND |
|---|---|
| 100.00 | Dimethicone |

Example 9: Finish Enhancing Composition 4

| AMOUNT | |
|---|---|
| (BY WEIGHT) | COMPOUND |
| 100.00 | Dimethylsiloxane methyl (polyoxytheylene) siloxane |

Example 10: Tinted Finish-Enhancing Composition

| | |
|---|---|
| AMOUNT | |
| (BY WEIGHT) | TINTED FINISH ENHANCING COMPOUND |

Add ˜1% thickening agent such as silicone and about 5% pigment to the composition of examples 6-10 to 2000 grams of an enhancing agent add 100 grams of pigment and 20 grams of silicone.

Examples 11-17 describe preferred embodiments of the cosmetic removing formulations.

Example 11: Remover

Ingredients in the remover are water, a soap or surfactant such as Disodim cocoamphodiacetate, a (organic or inorganic) salt such as trisodium phosphate and preservative such as phenoxyethanol and or ethylparaben, methylparaben and propylparaben and butylparaben. Alcohol can be substituted for the soap/surfactant or the preservative. If the alcohol is used as the soap/surfactant you do not need a preservative.

Example 12: Cosmetic Removing Formulation 1

| AMOUNT | |
|---|---|
| (BY WEIGHT) | COMPOUND |
| 90.29 | DI water |
| 1.78 | Trisodium phosphate |
| .20 | Polyoxytheylene polyoxypropylene |
| | glycol (Poloxamer 407) |
| .13 | Acesulfame K |
| 1.60 | Phenoxyethanol |
| 6.00 | Cocoampho acetate (Empigen CDR 30) |

To formulate the above composition, trisodium phosphate, Poloxamer, Acesulfame K are first added to warm water. The cocoampho acetate is then added to this aqueous composition, followed by the addition of phenoxyethanol.

The above formulation may also be used to remove coloring applied around the eyes.

Example 13: Cosmetic Removing Formulation 2

| AMOUNT | |
|---|---|
| (BY WEIGHT) | COMPOUND |
| 80.67 | DI water |
| 16.25 | Ethanol (denatured with flavor) 190 proof |
| 1.25 | Trisodium phosphate |
| .25 | Xanthan gum |
| .20 | Polyoxytheylene polyoxypropylene glycol |
| | (Poloxamer 407) |
| .15 | Sodium lauryl sulfate |
| .13 | Sodium saccharin |
| 1.0 | Sodium Benzoate |

To prepared the above formulation, trisodium phosphate, Poloxamer, sodium saccharine, sodium lauryl sulfate, and sodium benzoate are added to warm water. The xanthan gum is added separately to the alcohol. Next the alcohol mixture is added to the aqueous mixture. Finally, the glycerin is added to the above mixture.

Alternatively, the xanthan gum may be added to half of the water at room temperature. The trisodium phosphate, Poloxamer, sodium saccharin, sodium lauryl sulfate, and sodium benzoate may be added to warm water. The two aqueous mixtures may then be combined with the alcohol.

Example 14: Cosmetic Removing Formulation 3

| AMOUNT | |
|---|---|
| (BY WEIGHT) | COMPOUND |
| 80.02 | DI water |
| 16.25 | Ethanol (denatured with flavor) 190 proof |
| 1.25 | Trisodium phosphate |
| .25 | Xanthan gum |
| .20 | Polyoxytheylene polyoxypropylene glycol |
| | (Poloxamer 407) |
| .30 | Glycerin |
| .13 | Sodium saccharin |
| 1.60 | Phenoxyethanol |

To formulate the above composition, the trisodium phosphate, sodium saccharine, phenoxyethanol, and Poloxamer are added to warm water. The xanthan gum is separately mixed with the alcohol. The alcohol mixture is then combined with the aqueous mixture and the glycerin is added thereto.

Alternatively, the xanthan gum may be added to half of the water at room temperature. The trisodium phosphate, sodium saccharine, phenoxyethanol, and Poloxamer are added to warm water may be added to warm water. The two aqueous mixtures may then be combined with the alcohol.

Example 15: Cosmetic Removing Formulation 4

| AMOUNT | |
|---|---|
| (BY WEIGHT) | COMPOUND |
| 90.30 | DI water |
| 1.78 | Trisodium phosphate |
| .20 | Polyoxytheylene polyoxypropylene glycol |
| | (Poloxamer 407) |
| 6.00 | Cocoampho acetate (Empigen CDR 30) |
| .12 | Sodium saccharin |
| 1.60 | Phenoxyethanol |

To formulate the above composition, the trisodium phosphate, sodium saccharine phenoxyethanol, and Poloxamer are added to warm water. The xanthan gum is separately mixed with the alcohol. The alcohol mixture is then combined with the aqueous mixture and the glycerin is added thereto. Finally, the cocoampho acetate is added thereto.

The above formulation may also be used to remove color applied around the eyes.

Example 16: Cosmetic Removing Formulation 5

| AMOUNT | |
|---|---|
| (BY WEIGHT) | COMPOUND |
| 91.69 | DI water |
| 1.78 | Trisodium phosphate |
| .20 | Polyoxytheylene polyoxypropylene glycol |
| | (Poloxamer 407) |
| 6.00 | Cocoampho acetate (Empigen CDR 30) |
| .13 | Acesulfame K |
| .20 | Quaternium 15 |

To formulate the above compositions, the Quaternium 15 is first added to the water. The Poloxamer, trisodium phosphate, and Acesulfame K are then added thereto. Finally, the cocoampho acetate is added thereto.

Example 17: Cosmetic Removing Formulation 6

| AMOUNT | |
|---|---|
| (BY WEIGHT) | COMPOUND |
| 0.5 | Sodium lauryl sulfate |
| 90.3 | Distilled water |
| 0.2 | Quatemium 15 |

Example 18: Cosmetic Removing Formulation 7

| AMOUNT | |
|---|---|
| (BY WEIGHT) | COMPOUND |
| 1.78 | Trisodium phosphate |
| 98.22 | Ethyl alcohol |

Example 19: Color Enhancing Composition

| AMOUNT (BY WEIGHT) | COMPOUND |
|---|---|
| 4.24 | Zinc Stearate |
| 3.74 | Bismuth oxychloride |
| 66.03 | Colored mica powder |
| 14.95 | C12-C15 alkyl benzoate |
| 8.72 | Synthetic wax/corn glutin protein mixture |
| | from Presperse, Inc., 601 Hadley Rd., South |
| | Plainfield, NJ 07080 |
| 1.3 | Ethylparaben |
| 0.65 | Methylparaben |
| 0.15 | Propylparaben |

To press the above composition, the loose powder is first blended in an industrial blender. The resulting composition is thereafter placed in a press.

Example 20: A Waxless Lip Balm

A waxless lip balm formulation is to add about 4-8% (preferred 5%) of a thickening agent such as silica (300 grams) to 4000 grams of an enhancing agent as disclosed herein. Silica functions as an ant caking agent, bulking agent, opacifying agent and suspending agent, any chemical that can provide any of the aforementioned functions will work. An enhancing agent formulation may be used.

Example 21: A Tinted Waxless Lip Balm

A tinted waxless lip balm formulation is to add pigment (about 0.01-25%) and a thickening agent such as silica (about 0.01-25%) to a formulation for an enhancing agent disclosed herein. For example, take 4000 grams of enhancing agent and add 280 grams of silica and 100 grams of pigment. Color can be created not just by adding pigment, but by using additives of natural extracts, for example, pomegranate, aloe leaves, and grape seeds. Regardless of additives, the silica level should remain about the same. Sunscreen can also be added.

Example 22: Use of Capsules

The capsules can be added to colored or clear formulations. The capsules can be purchased separately, they can also be injected with a dropper or syringe or through a cartage system such as a pen cartage. (one cartage of clear formula piercing the other cartage with the desired color or vitamin or whatever is desired in it in a pen device that mixes the two and flows through the pen).

Undoubtedly, numerous variations and modifications of the invention will become readily apparent to those familiar with cosmetic products. Accordingly, the scope of the invention should not be construed as limited to the specific examples described, because those examples are presented herein only as being illustrative of the many formulations possible according to the invention.

TABLE 1

| Compound | Suspension | Color | Rub | Drying | Feel on | Overall | |
|---|---|---|---|---|---|---|---|
| Number | Test | Test | Test | Time | Lips | Performance | Notes |
| 1 Resin 28- | Good | Good | Good | Good | Fair | Good | Versatile but can |
| 2930 | | | | | | | shrink/dry on lips |
| 2. | Good | Good | Good | Good | Good | Good | Best Resin (most) |
| Amphomer | | | | | | | preferred) |
| Lv-71 | | | | | | | |
| 3. Water | Fair | Fair | Poor | Fair | Poor | Poor | Creates nice film but too |
| Lock G40- | | | | | | | grainy |
| A180-D242 | | | | | | | |
| 4 Dathold | Fair | Poor | Fair | Poor | Fair | Fair | Similar to Amphomer |
| 5 Eastman | Fair | Fair | Fair | Fair | Fair | Fair | Similar to Amphomer |
| AQ-385 | | | | | | | |
| and AQ-555 | | | | | | | |
| 6. Ultra | Fair | Poor | Fair | Fair | Fair | Fair | Similar to Amphomer |
| Hold 8 | | | | | | | |
| 7 Omnirez | Good | Poor | Fair | Good | Good | Fair | Creates good film but |
| 2000 | | | | | | | poor color |
| 8 Gantarex | Poor | Poor | Poor | Fair | Poor | Poor | Burns lips, poor |
| compound | | | | | | | performance in rub test |
| 9. H2old | Fair | Poor | Poor | Good | Fair | Fair | Very sheer, does not hold |
| EP-1 | | | | | | | color well, felt good on |
| Terpolymer | | | | | | | lips and could be added to |
| | | | | | | | amphomer to strengthen |
| | | | | | | | hold |
| 10 | Good | Good | Good | Good | Good | Good | Good in every respect but |
| Amphomer | | | | | | | does not last as long on |
| Lovocryl-47 | | | | | | | lips and in rub tests |
| 11 | Good | Good | Fair | Good | Good | Good | Does not last as long in |
| Amphomer | | | | | | | rub tests |
| 28-4910 | | | | | | | |
| 12 | Fair | Poor | Poor | Good | Poor | Fair | Causes puckering of lips, |
| Advantage | | | | | | | too tight, too firm to pass |
| Plus | | | | | | | rub tests; comes off in |
| terpolymer | | | | | | | patches on lips, dries |
| | | | | | | | quickly, aids in a tack free |
| | | | | | | | feeling on lips |
| 13 | Poor | Poor | Fair | Good | Poor | Poor | Tacky on lips, transparent |
| Copolymer | | | | | | | color; too thin; resin may |
| 958 | | | | | | | be too firm |
| 14 Joneyrl | Poor | Fair | Poor | Good | Fair | Fair | Burned lips, tranparent |
| | | | | | | | color, doesnt' pass rub |
| | | | | | | | test, dried quickly, left |
| | | | | | | | sediment in bottom of |
| | | | | | | | solution |
| 15. Sentyr | Poor | Poor | Poor | Poor | Fair | Poor | Hard to work with in |
| Polyvinyl | | | | | | | adding other ingredients |
| acetate-12 | | | | | | | slow drying time; sticky |
| | | | | | | | on lips |
| 16. | Fair | Poor | Fair | Poor | Fair | Fair | |
| Carboset- | | | | | | | |
| 525 | | | | | | | |

I claim:

1. A waxless lip balm comprising an enhancing agent and a thickening agent.

2. The waxless lip balm of claim 1, further comprising a coloring agent.

3. The waxless lip balm of claim 1 wherein the thickening agent is silica.

4. A tinted enhancing agent comprising an enhancing agent, a thickening agent, and a coloring agent.

5. A kit comprising a plurality of compositions, selected from the group consisting of a film-forming agent, an enhancing agent, a lip coloring agent and a remover.
